(19) United States
(12) Patent Application Publication
(10) Pub. No.: US 2017/0128638 A1
(43) Pub. Date: May 11, 2017

(54) MEDICAL SUCTION PUMP AND FLUID COLLECTING VESSEL

(71) Applicant: MEDELA HOLDING AG, Baar (CH)

(72) Inventors: Charles Giezendanner, Morschach (CH); Martin Melzer, Cham (CH); Martin Walti, Zurich (CH)

(21) Appl. No.: 15/318,648

(22) PCT Filed: Jun. 18, 2015
(86) PCT No.: PCT/EP2015/063712

§ 371 Date: Dec. 13, 2016

(30) Foreign Application Priority Data
Jun. 26, 2014 (EP) 14174595.0

(51) Int. Cl. A61M 1/00; A61M 1/36; A61M 1/02

(52) U.S. Cl. CPC A61M 1/0025; A61M 1/0001; A61M 1/0068; A61M 1/024; A61M 1/3666; A61M 2205/3379; A61M 2205/75; A61M 2205/3317

(57) ABSTRACT

A medical suction pump for aspiration of bodily fluid has a suction pump housing, a fluid collection container held releasably on the housing, and at least one filling level sensor. The fluid collection container has a first side wall and at least one further side wall which together delimit at least one interior space for receiving the aspirated fluid. The at least one filling level sensor extends at least approximately parallel to the first side wall over at least a partial area of the fillable height of the interior space. The first side wall is place at least in part, and the cross section of the at least one interior space tapers vertically from the top downwards, at least over a partial area of the fillable height.

### CROSS-REFERENCE TO RELATED APPLICATIONS

The present application is the US national phase of International Patent Application No. PCT/EP2015/063712, filed Jun. 18, 2015, which application claims priority to European Application No. EP 14174595.0, filed Jun. 26, 2014. The priority application EP 14174595.0, is hereby incorporated by reference.

### TECHNICAL FIELD

The present invention relates to a medical suction pump and to a fluid collection container of such a suction pump.

### PRIOR ART

Medical suction pumps for aspiration of bodily fluid are used in various sectors, for example during or after surgical interventions, in wound drainage, thorax drainage or liposuction. These vacuum pump systems usually have a vacuum pump, one or more fluid collection containers, and a drainage hose connection between patient and fluid collection container.

By means of a vacuum pump, an underpressure is generated in the fluid collection container, as a result of which the fluid or secretions from a cavity of the patient can be aspirated through the drainage hose into the container and can be collected there. WO 2007/128156 and WO 2008/141471 disclose medical suction pumps of this kind in which the fluid collection container is secured pivotably and releasably on a pump housing. WO 2011/054118 and WO 2012/097462 propose arranging a capacitive filling level sensor on the housing in order to determine the filling level in the fluid collection container. Although these filling level sensors have proven useful, they are not really able to determine small filling level quantities.

Medical suction pumps and fluid collection containers are also used in mechanical autotransfusion, in particular for heart-lung machines.

Moreover, EP 1 589 325 and WO 2009/098077 disclose open jugs in which consumption of liquid can be determined. For this purpose, the jugs are arranged in holders that are provided with filling level sensors. The jugs have a conical main shape and their holders are designed accordingly. The filling level sensors extend along an oblique wall of the holder.

### DISCLOSURE OF THE INVENTION

It is an object of the invention to make available a medical suction pump and an associated fluid collection container in which, by simple means, it is possible to detect a filling level ranging from a small filling quantity to the approximately filled fluid collection container.

In order to achieve this object, the medical suction pump according to the invention for aspiration of bodily fluid has a suction pump housing, a fluid collection container held releasably on the housing, and at least one filling level sensor for detecting a filling level of the fluid collection container. The fluid collection container has a first side wall and at least one further side wall, which together delimit at least one interior space for receiving the aspirated fluid, wherein the interior space has a height that is fillable with fluid. The at least one filling level sensor extends at least approximately parallel to the first side wall over at least a partial area of the fillable height. The first side wall is plane at least in part, and the cross section of the at least one interior space tapers vertically from the top downwards, at least over a partial area of the fillable height.

In this connection, “at least in part” means discernible to the eye and not infinitesimal.

In a preferred embodiment, the filling level sensor extends parallel to the first side wall and/or the first side wall is continuously plane or plane in steps. These steps preferably extend in the horizontal direction.

By virtue of the tapering of the cross section of the interior space in the lower area of the container, i.e. at the place where the container is filled first, the filling level rises more quickly with a small filling quantity than with a larger filling quantity. In this way, a change of the filling quantity in the lowermost area of the container is associated with a greater change of the filling height than in the upper area. Compared to a container with a constant cross section, the measurement accuracy can therefore be hugely increased, without the sensor itself having to be modified.

Preferably, the first side wall extends in the vertical direction. This simplifies the calculation of the filling level quantity and, if appropriate, of the filling speed or filling rate based on the sensor signal.

Preferably, the first side wall is arranged on the side towards the filling level sensor, or on a side of the fluid collection container directed away from the at least one filling level sensor. This also facilitates the calculation and increases the measurement accuracy as a whole.

Preferably, the at least one filling level sensor has a shape corresponding to the shape of the first side wall, such that the first side wall has a constant distance from the at least one filling level sensor over the entire length of the at least one filling level sensor or bears on the filling level sensor over the entire length of the at least one filling level sensor. A constant sensor signal is thereby ensured.

Preferably, the at least one filling level sensor is arranged on the suction pump housing. Preferably, the at least one filling level sensor is a capacitive filling level sensor. Examples of such suction pump housings and of such sensors are disclosed in WO 2011/054118 and WO 2012/097462. The disclosure of these two publications is herewith incorporated into the present text.

In particular, the fluid collection container can be held on the suction pump housing in such a way as to be able to pivot in and out, in which case it can be completely releasable and can be removed from the suction pump housing.

If the filling level sensor is arranged in or on a wall of the suction pump housing, this wall preferably has a shape corresponding to the shape of the first side wall, such that the first side wall is at a constant distance from the wall of the suction pump housing over the entire length of the at least one filling level sensor or bears on the wall of the suction pump housing over the entire length of the at least one filling level sensor.

Alternatively, a filling level sensor of this kind or another filling level sensor can also be arranged on the fluid collection container or can be integrated in one of its walls, in particular into the first side wall. Here too, the shape of the filling level sensor preferably follows that of the surface of the first side wall of the fluid collection container, which is directed towards the inside of the container.

The associated fluid collection container according to the invention has a first side wall and at least one further side wall, which together delimit at least one interior space for receiving the aspirated fluid, wherein the interior space has a height that is fillable with fluid. The fluid collection container moreover has at least one suction port and at least one fluid port, which permit a connection to the interior space. The first side wall is plane at least in part, and the cross section of the at least one interior space tapers vertically from the top downwards, at least over a partial area of the fillable height.

The side walls surround the interior space. Depending on how the side walls are configured, a floor is also present, or they adjoin each other such that no floor is needed to close the container at the bottom. Preferably, an upper wall is also present. The side walls, if appropriate together with the floor and the wall, preferably surround the interior space of the container in such a way as to form a container that is closed except for a few connection openings.

The first side wall preferably has a constant thickness. Preferably, the other walls also have constant thicknesses. All the side walls are preferably formed substantially by plane surfaces.

The tapering of the interior space is preferably such that a sufficient measurement or detection accuracy can be achieved. Preferably, a lower cross-sectional area of the interior space is less than or equal to a quarter, preferably less than or equal to an eighth, of an upper cross-sectional area of the interior space.

In order to make the calculation and measurement easier, preferably only one side wall extends at an inclination with respect to the vertical direction, and all the other side walls extend in the vertical direction. This inclined side wall can be the first side wall or one of the other side walls. Preferably, it is one of the other side walls. The inclination is preferably rectilinear, i.e. formed by a oblique plane.

In a preferred embodiment, one of the at least one further side wall is a second side wall which extends at an inclination with respect to the vertical direction at least over said partial area of the fillable height. It thus narrows the cross section.

In one embodiment, the fluid collection container has a third and a fourth side wall adjoining the first side wall, wherein the second side wall lies opposite the first side wall, and wherein the third and fourth side walls are plane and extend in the vertical direction. In this embodiment, the third and fourth side walls preferably each form an acute triangle in said partial area.

In another embodiment, the second side wall adjoins the first side wall, wherein a third side wall, which lies opposite the second side wall, adjoins the first side wall, and wherein a fourth side wall is present which lies opposite the first side wall. The first, second, third and fourth walls enclose one of the at least one interior spaces, wherein the third and fourth walls are plane and extend in the vertical direction. In this embodiment, the collection container preferably has two interior spaces, which are separated completely from each other by a separating wall, wherein the separating wall extends in the vertical direction and forms the third wall.

Preferably, the suction port is arranged in the first side wall. This is preferably the same wall that is directed towards and nearest to the at least one filling level sensor. The suction pump is preferably an electrically operated vacuum pump, in particular a piston pump or a diaphragm pump.

### BRIEF DESCRIPTION OF THE DRAWINGS

Preferred embodiments of the invention are described below with reference to the drawings, which serve only for illustration and are not to be interpreted as limiting the invention. In the drawings:

FIG. 1 shows a perspective view of a suction pump according to the invention, with a fluid collection container according to the invention in a first embodiment;

FIG. 2 shows the suction pump according to FIG. 1, with the fluid collection container pivoted out;

FIG. 3 shows a perspective view of a variant of the fluid collection container according to FIG. 1;

FIG. 4 shows a side view of the fluid collection container according to FIG. 3;

FIG. 5 shows a side view of a fluid collection container according to the invention in a second embodiment;

FIG. 6 shows a side view of a fluid collection container according to the invention in a third embodiment;

FIG. 7 shows a perspective view of a fluid collection container according to the invention, with the interior space made visible, in a fourth embodiment;

FIG. 8 shows a first longitudinal section through the fluid collection container according to FIG. 7;

FIG. 9 shows a second longitudinal section through the fluid collection container according to FIG. 7, and

FIG. 10 shows a cross section through the fluid collection container according to FIG. 7.

### DESCRIPTION OF PREFERRED EMBODIMENTS

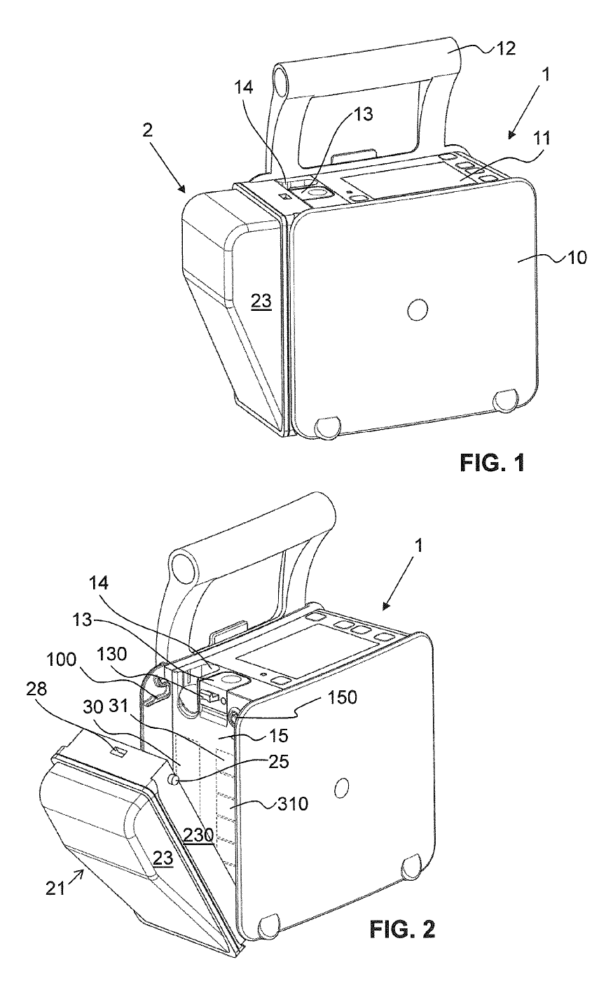

FIGS. 1 and 2 show a drainage pump unit, the basic design of which is known from WO 2007/128156 and WO 2008/141471, for example. These pump units are used for aspiration of bodily liquids or bodily fluids in the medical field and are preferably portable. In other words, the patient is able to move around freely during the pumping.

The drainage pump unit has a suction pump housing 1, with a fluid collection container 2 secured releasably thereon and completely removable from the housing 1. The suction pump housing 1 and the fluid collection container 2 are preferably made of plastic.

The pump assembly and control electronics are arranged in the housing 1. In this example, the housing is cuboid. However, it can also have other shapes.

A display and operating panel 11 is arranged in an upper wall of the housing 1. A handle 12 makes transport easier. Alternatively or in addition to the carrying handle 12, the housing 1 preferably has a support surface or support feet, such that the housing 1, in the position depicted, can be placed on a table or on another suitable support surface. Indications made below regarding directions are to be interpreted in this standing position of the housing.

A front wall visible in the figures is designated by reference sign 10, and a side wall by 15. The visible front wall 10, and a rear wall lying opposite the latter and extending parallel thereto, protrude from the side wall 15. They form a seat for the fluid collection container 2.

The upper wall has a recess 14 in the area of the side wall 15. Moreover, a locking element 13 is also present there, which has a latching lug 130. This locking element 13 serves to releasably fix the fluid collection container 2 on the housing 1. For this purpose, the fluid collection container 2 preferably has a recess 28.

The fluid collection container 2 is preferably pivoted into the housing 1 and latches in this position. For this purpose, the protruding parts of the front wall 10 and of the rear wall have upper and lower slotted guides 100 or latching apertures, into which lower and upper latching pins 24, 25 of the fluid collection container 2 engage.

Instead of slotted guides, latching pins and latching hooks, the collection container 2 can also be secured and fixed by other means. The physical configuration of the housing 1 and of the collection container 2 in the area where they adjoin each other can also be different.

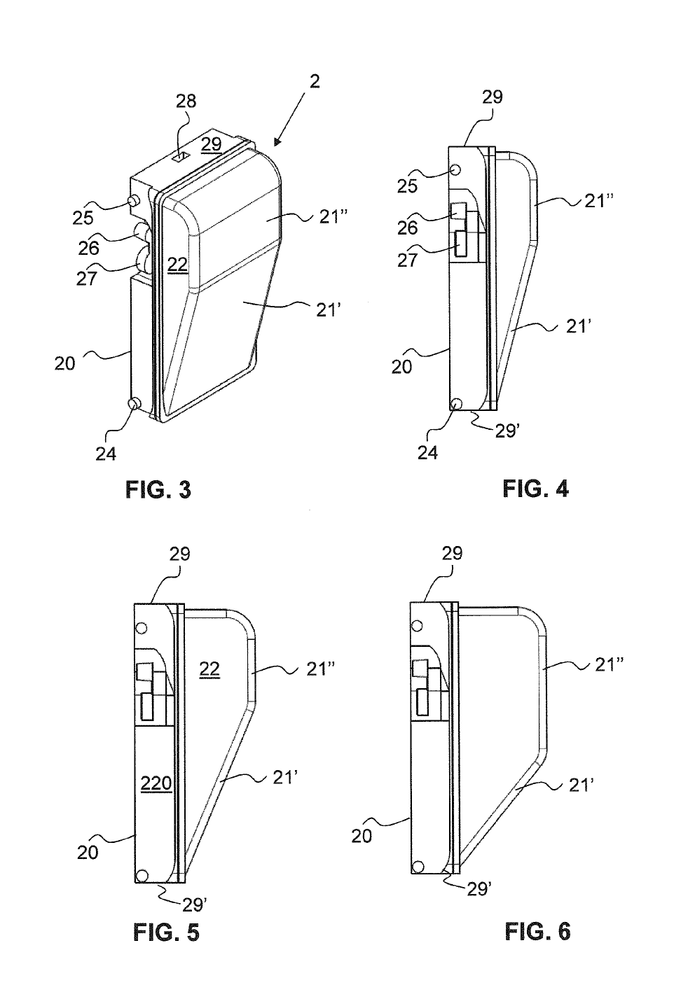

The fluid collection container has a first side wall 20, which extends in the vertical direction and is plane. This side wall 20 can be clearly seen in FIGS. 3 and 4. FIGS. 3 and 4 show a container 2 which is designed the same way as the container 2 shown in FIGS. 1 and 2. The only difference is that the openings in its first side wall 20 are not arranged at the same location. Thus, in the container 2 according to FIGS. 1 and 2, a drainage port for connection to an adapter part on the patient side is located on a left-hand side of the first side wall 20, a vacuum port for connection to the suction pump is located on a right-hand side of the first side wall 20, and a discharge opening for emptying the container 2 is likewise located on the right-hand side. By contrast, in the illustrative embodiment according to FIGS. 3 and 4, the drainage port is located on the right-hand side and the vacuum port 26 and the discharge opening 27 are located on the left-hand side. Accordingly, the corresponding mating pieces in the pump housing 1, i.e. a vacuum port 150 and the recess 14 for receiving an adapter part on the patient side, are also arranged differently than shown in FIGS. 1 and 2. In the following, reference is made to FIGS. 3 and 4 when the container according to FIGS. 1 and 2 is described.

With the abovementioned first side wall 20, the fluid collection container 2 bears on the side wall 15 of the suction pump housing 1, wherein a small air gap may be present. The side wall 15 of the housing 1 is likewise plane and extends in the vertical direction. In each case, a rectangular portion 220, 230 of two opposite side walls 22, 23 adjoining the first side wall 20 is received between the protruding front wall 10 and the rear wall of the housing 1.

The housing-side vacuum port 150 is arranged in the side wall 15 of the housing 1. It is preferably arranged in an upper area of the side wall 15. When the container 2 is pivoted in, the vacuum port 150 is connected to the vacuum port 26 of the container 2 shown in FIGS. 3 and 4.

An adapter part of a patient-side drainage hose can be inserted into the recess 14 and, by pivoting the container 2 inwards, can be connected to the drainage port (not visible in the figures) of the container 2. This is known from WO 2008/141471, for example.

Reference number 27 indicates the discharge opening which is closed during use of the container 2 and is opened only to empty the filled container 2 when the latter has been removed from the pump housing 1.

By way of the vacuum port 150, an underpressure is generated in the container 2 by means of the suction pump. By virtue of this underpressure, a fluid or secretion is aspirated from the cavity of the patient through a drainage tube and, through the drainage tube, into the container 2 and collected there.

The suction pump moreover comprises at least one capacitive filling level sensor, by means of which the filling level in the fluid collection container 2 can be determined. In this example, the filling level sensor is arranged in the side wall 15 of the housing facing towards the fluid collection container 2.

The sensor has at least two electrodes, in this case precisely two electrodes 30, 31, which are arranged at a distance from each other. A first electrode 30 is configured in one piece and forms a transmitter. A second electrode 31 is segmented and forms a receiver for receiving the signal sent from the transmitter. A charge transfer from the transmitter electrode to the receiver electrode is determined. The two electrodes 30, 31 extend parallel to and at a distance from each other. In this example, they are arranged in a common plane. Their transmitter and receiver surfaces, respectively, are thus oriented in the same direction, namely towards the fluid collection container 2. This filling level sensor is described in WO 2012/097462. Alternatively, another filling level sensor can also be used, preferably a capacitive filling level sensor. The filling level sensor, here formed by the two electrodes 30, 31, is band-shaped and extends vertically over a desired measurement area of the container 2, preferably over the filling height that is to be measured and detected.

As can be seen in FIGS. 1 to 4, the container 2 according to the invention has a downwardly tapering cross section. For this purpose, the third side wall 22 and the fourth side wall 23 have a portion which is designed as an acute triangle. Correspondingly, the second side wall 21 lying opposite the first side wall 20 is inclined. It preferably has a portion 21′, inclined with respect to the vertical direction, and a vertically extending middle portion 21″. An upper portion preferably has a curved design and merges into an upper wall 29. A floor 29′ of the container 2 is correspondingly smaller in relation to the upper wall 29.

The interior space of the container 2 is designed corresponding to the shape of these walls. Preferably, the walls therefore have a constant thickness.

FIGS. 5 and 6 show a second illustrative embodiment and a third illustrative embodiment of the container 2. The interior space has a greater volume than the container according to FIGS. 3 and 4. However, these containers 2 can be pivoted into and held in the same housing 1 according to FIGS. 1 and 2. As can be seen by a comparison of FIGS. 4 to 6, they differ from each other only in terms of the angle of inclination and the length of the lower portion 21′ of the second side wall 21 and, therefore, in the shape of the third side wall 22 and fourth side wall 23.

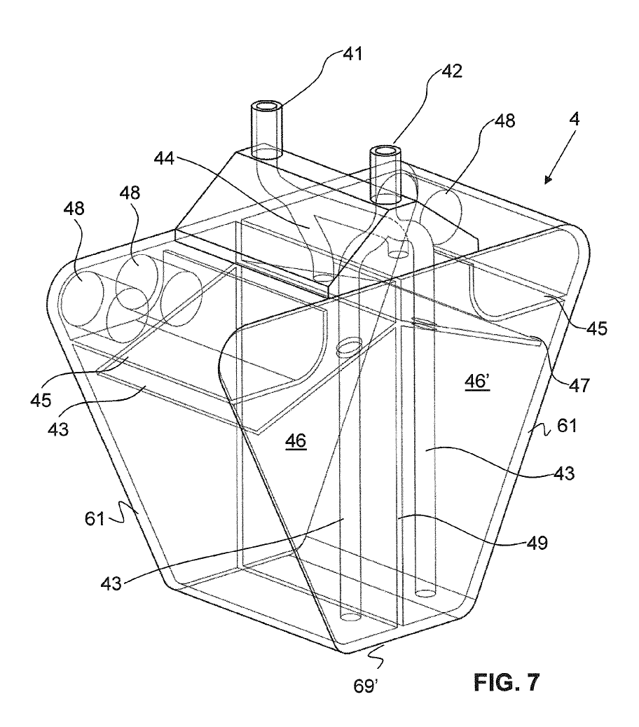

FIGS. 7 to 10 show a fourth illustrative embodiment of a fluid collection container 4 according to the invention. This container can be held in a differently shaped suction pump housing. However, it is once again also possible for it to be held pivotably, releasably and completely removably, for example by providing it with corresponding pins. This container is particularly suitable for use in an autotransfusion appliance for receiving and transferring blood, for example to a heart-lung machine.

This fluid collection container 4 has two chambers 46, 46′ completely separate from each other, and therefore two interior spaces independent of each other. Two filling level sensors (not shown here) are accordingly present. For example, it is possible to use the above-described filling level sensors, in particular two electrode pairs that each comprise two band-shaped electrodes spaced apart from each other.

During use in an autotransfusion appliance, the chambers serve to collect blood. It is therefore blood that is referred to below, even though the container is also suitable for collecting another bodily fluid.

The container 4 is preferably in one piece. It is preferably made of a plastic. Like the containers 2 already mentioned above, it is preferably also transparent and preferably designed as a disposable product and discarded after use.

On its top face, the container 4 has a patient-side port 41 and a transfer-side port 42. Otherwise, the container 4 has a mirror symmetrical configuration. From the patient-side port 41, a branching connection line 44 having a Y shape leads into the interior space of the container. From a transfer-side port 42, a Y-shaped branch leads into two ascending pipes 43, which reach into the lower area of the interior space of the container.

The interior space of the container 4 is divided into two areas, which are preferably completely separate from each other. In this example, the areas are located next to each other and are separated from each other by a separating wall 49. One of said connection lines 44 and one of said ascending pipes 43 lead in each case into one of the areas. An individual area is described below. The other area is of identical construction.

The area is basically formed by a blood collection chamber 46 which, in its upper area, is delimited by an oblique rib 47. Starting from the separating wall 49, this oblique rib 47 extends down towards the opposite side and in so doing it extends almost but not quite completely over the full width of the chamber. In the direction perpendicular thereto, the oblique rib 47 extends over the entire length of the area or of the chamber 46. The ascending pipe 43 passes through the oblique rib 47.

Arranged in the area above the oblique rib 47 is the upwardly curved separating rib 45, which extends over the entire length of the area and defines a pump-side area. This area is connected to the rest of the interior space only by a narrow upper gap. In this area, a first connector opening 40 and a second connector opening 40′ are arranged, which serve for connection to a vacuum line or an overpressure line of the suction pump. These connector openings 40, 40′ are provided with filters 48. These filters 48 are known in the prior art. Other filters can also be present at other locations, as is likewise known from the prior art. These filters serve to protect the appliance.

The separating rib 45 prevents blood from spraying onto the filters and possibly closing them early. Moreover, it forms a further protection for the pumps. The oblique rib 47 prevents the aspirated blood from sloshing back into the suction line and additionally allows aspirated blood to flow down via the oblique rib 47 into the collection chamber 46.

The chamber 46 has a downwardly tapering cross section. Preferably, the surface area in the lowermost region of the chamber 46 is at most half the surface area in the upper region, i.e. in the region just below the lower end of the oblique rib 47. This permits a relatively precise measurement of the filling level even with small filling quantities, particularly when capacitive filling level sensors are used for this purpose.

Preferably, the blood collection container 4 therefore has a vertically extending, plane and trapezoid first side wall 60, two obliquely extending second side walls 61 lying opposite each other, a third side wall in the form of the separating wall 49 extending vertically between the two second side walls 61, and a likewise trapezoid fourth side wall 63 lying opposite the first side wall 60 and extending in a plane parallel thereto. An upper wall bears the reference sign 69, while a floor at the bottom bears the reference sign 69′.

The filling level sensors are preferably arranged adjacent to the first side wall 60 in the corresponding chambers 46, 46′. The sensors are also preferably arranged on the housing and, in terms of their shape, extend in a manner corresponding to the first side wall 60. However, they can also be arranged on or in the first side wall.

The two chambers 46 of the container 4 can be filled and emptied alternately. That is to say, when one chamber is being filled, the other can at the same time be emptied. Emptying takes place at the latest when the corresponding filling level sensor of a control unit of the appliance indicates that the corresponding chamber is full.

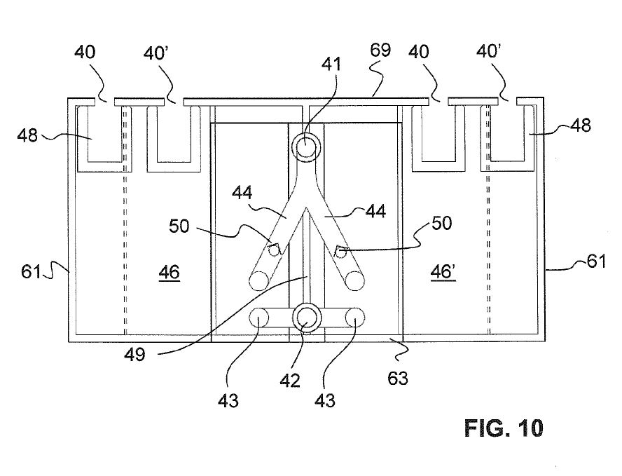

One-way valves 50 are accordingly present which open and close the connection between the suction line and the collection chamber 46. These valves can be controlled by the control unit or can close at a predetermined pressure. As is shown in FIGS. 9 and 10, they can be part of the blood collection container 4, or they can also be arranged in the area of the suction line or at the patient-side port.

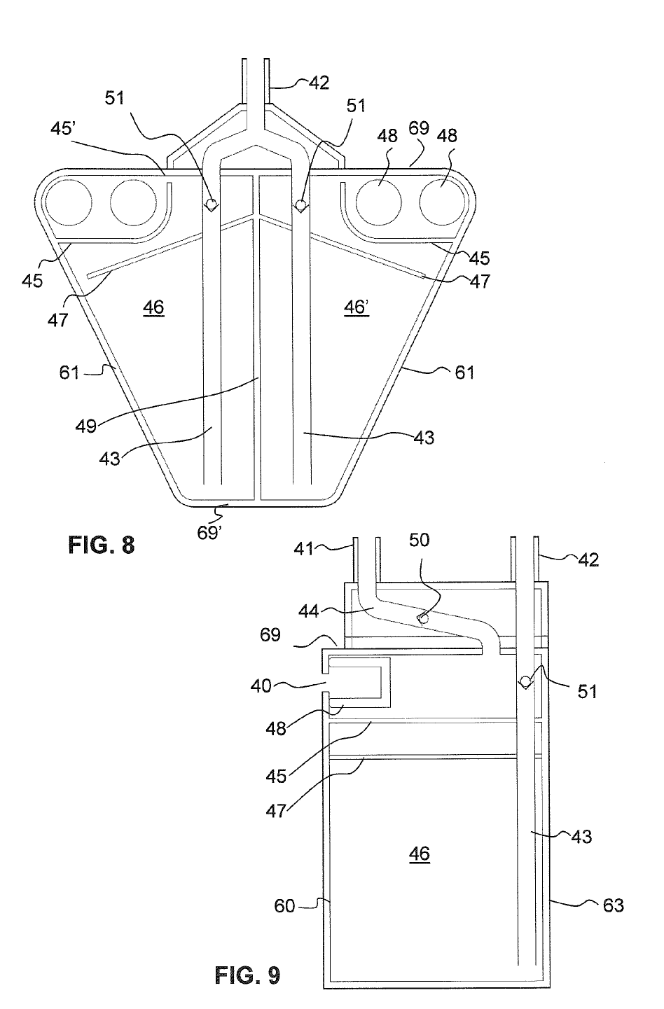

Moreover, one-way valves 51 are present which open and close the connection between the collection chamber 46 and the transfer line. These valves can likewise be controlled by the control unit or can close at a predetermined pressure. As is shown in FIGS. 8 and 9, they can be part of the blood collection container 4, or they can also be arranged in the area of the transfer line or at the transfer-side port 42.

The one-way valves can be simple non-return valves, for example, or they can likewise be controlled by the control unit.

The suction pump according to the invention and the fluid collection container according to the invention permit a relatively precise detection of a filling level even at a low filling level height.

What is claimed is:

1. A medical suction pump for aspiration of bodily fluid, wherein the suction pump has a suction pump housing, a fluid collection container held releasably on the housing, and at least one filling level sensor for detecting a filling level of the fluid collection container,
wherein the fluid collection container has a first side wall and at least one further side wall, which together delimit at least one interior space for receiving the aspirated fluid,
wherein the interior space has a height that is fillable with fluid,
wherein the at least one filling level sensor extends at least approximately parallel to the first side wall over at least a partial area of the fillable height,
wherein
the first side wall is planar at least in part, and
wherein the cross section of the at least one interior space tapers vertically from the top downwards, at least over a partial area of the fillable height.

2. The suction pump according to claim 1, wherein the first side wall extends in the vertical direction.

3. The suction pump according to claim 1, wherein the first side wall is arranged on the side towards the filling level sensor, or on a side of the fluid collection container directed away from the at least one filling level sensor.

4. The suction pump according to claim 1, wherein the at least one filling level sensor has a shape corresponding to the shape of the first side wall, such that the first side wall has a constant distance from the at least one filling level sensor over the entire length of the at least one filling level sensor or bears on the at least one filling level sensor over the entire length of the at least one filling level sensor.

5. The suction pump according to claim 1, wherein the at least one filling level sensor is arranged on the suction pump housing.

6. The Suction pump according to claim 1, wherein the at least one filling level sensor is a capacitive filling level sensor.

7. A fluid collection container of a medical suction pump, wherein the suction pump serves for aspiration of bodily fluid and has and at least one filling level sensor for detecting a filling level of the fluid collection container, wherein the fluid collection container has a first side wall and at least one further side wall, which together delimit at least one interior space for receiving the aspirated fluid, wherein the interior space has a height that is fillable with fluid,
wherein the fluid collection container moreover has at least one suction port and at least one fluid port, which permit a connection to the interior space,
wherein
the first side wall is plane at least in part, and
the cross section of the at least one interior space tapers vertically from the top downwards, at least over a partial area of the fillable height.

8. The fluid collection container according to claim 7, wherein all the side walls are formed substantially by plane surfaces.

9. The fluid collection container according to claim 8, wherein only one of the side walls extends at an incline with respect to the vertical direction, and all the other side walls extend in the vertical direction.

10. The fluid collection container according to claim 7, wherein one of the at least one further side wall is a second side wall which extends at an incline with respect to the vertical direction at least over said partial area of the fillable height.

11. The fluid collection container according to claim 10, wherein the fluid collection container has a third and a fourth side wall adjoining the first side wall, and wherein the second side wall lies opposite the first side wall, wherein the third and fourth side walls are planar and extend in the vertical direction.

12. The fluid collection container according to claim 11, wherein the third and fourth side walls each form an acute triangle in said partial area.

13. The fluid collection container according to claim 10, wherein the second side wall adjoins the first side wall, and wherein a third side wall, which lies opposite the second side wall, adjoins the first side wall, and wherein a fourth side wall is present which lies opposite the first side wall, wherein the first, second, third and fourth walls enclose one of the at least one interior spaces, wherein the third and fourth walls are plane and extend in the vertical direction.

14. The fluid collection container according to claim 13, wherein the collection container has two interior spaces, which are separated completely from each other by a separating wall, wherein the separating wall extends in the vertical direction and forms the third wall.

15. The fluid collection container according to claim 7, wherein the suction port is arranged in the first side wall.
